(19) United States
(12) Patent Application Publication
(10) Pub. No.: US 2025/0275798 A1
(43) Pub. Date: Sep. 4, 2025

(54) Femoral Neck Fracture Fixation Device

(71) Applicant: Joseph Patterson, Los Angeles, CA (US)

(72) Inventor: Joseph Patterson, Los Angeles, CA (US)

(21) Appl. No.: 18/969,046

(22) Filed: Dec. 4, 2024

Related U.S. Application Data
(60) Provisional application No. 63/561,229, filed on Mar. 4, 2024.

(51) Int. Cl. A61B 17/88; A61B 17/16; A61B 17/17

(52) U.S. Cl. CPC A61B 17/8872; A61B 17/1668; A61B 17/1728

(57) ABSTRACT

Systems and devices for bone fracture fixations are described. The system enables the closed reduction of bone fracture fixation by means of a conformable fixed-angle fracture fixation device. The fixed angle fracture fixation device utilizes a series of fixation elements to secure the fracture at multiple fixed angles reducing the risk of nonunion, implant failure and the likelihood for patients to undergo subsequent operations.

### CROSS-REFERENCE TO RELATED APPLICATIONS

This application claims priority to U.S. Patent Application No. 63/561,229, filed Mar. 4, 2024, the disclosures of which are incorporated herein by reference in their entirety.

### FIELD OF THE INVENTION

This application generally relates to bone fixation assemblies. More specifically, femoral fracture fixation assemblies, kits for bone fixation assemblies, methods of assembling bone fixation assemblies, and methods of implanting fixation assemblies in a bone.

### BACKGROUND

Implants are medical devices manufactured to replace a missing biological structure, support a damaged biological structure, or enhance an existing biological structure. For example, an implant may be a rod, used to strengthen weak bones. In a broader sense, an implant can be any object that is inserted or grafted into the body. This could include a wide range of medical devices such as pacemakers, joint replacements, dental implants, and even cosmetic implants.

Orthopedic surgery is a branch of medicine that focuses on diagnosing, treating, repairing, and preventing conditions affecting the musculoskeletal system, including bones, joints, ligaments, tendons, muscles, and nerves. Orthopedic surgeons, also known as orthopedists, are medical specialists who are trained to diagnose and treat issues related to the musculoskeletal system. Orthopedic surgeons can perform a broad range of procedures, including ankle, knee, hip, spinal, hand, and neck surgeries. Typically, they'll attempt to treat an issue in the most holistic and least intrusive way possible, which is better for the healing and recovery of the patient. Common types of orthopedic surgery include joint replacement, fracture care and trauma, sports injuries, limb deformity, and surgeries for bone cancers.

A femoral neck fracture is a common injury to the proximal femur, which is associated with an increased risk of avascular necrosis and impaired fracture healing, and high levels of patient morbidity and mortality. The femoral neck is located in the upper portion of the femur, just below the ball part (femoral head) of the ball-and-socket joint (hip joint). Fractures in this area are common and can occur due to high-energy impacts in young patients or low-energy falls in older patients. The treatment is generally operative with open reduction and internal fixation. For open reduction, the surgeon makes an incision to access the affected bones and reset them to their proper place. Internal fixation refers to how the surgeon keeps the bone in place while it heals.

Orthopedic surgeons often keep bones in place for healing by using hardware such as plates, screws, nails, rods, or wires and pins. The type of hardware used depends on the location and type of fracture. Plates act like internal splints that hold the broken pieces of bone together. They are attached to the bone with screws. Screws are used for internal fixation more often than any other type of implant. They are either used alone for bone-to-bone and bone-to-tissue fixation or with implants like orthopedic plates. For some fractures of the long bones, the best way to hold the bone pieces together is by inserting a rod or nail through the bone. Typically screws at each end of the rod are used to keep the fracture from shortening or rotating and hold the rod in place until the fracture has healed. Wires and pins are often used to pin the bones back together. They are often used to hold together pieces of bone that are too small to be fixed with screws.

The purpose of hardware and other implants for fracture fixation is to restore the bone anatomy, provide stable fixation, accelerate recovery, and ensure more predictable and potentially faster healing.

### SUMMARY OF THE INVENTION

Devices and methods in accordance with some embodiments of the invention are directed to a bone fixation system.

Many embodiments of the disclosure are directed to a bone fracture fixation device comprising, a plate, a primary fixation element, at least one supplemental fixation element, and at least one positioning fixation element; wherein the plate tappers at an end and is further configured with at least one hole to receive the at least one positioning fixation element; and wherein the plate is configured to conform to a bone for fixation, for insertion without open exposure of a fracture site, and to receive the primary fixation element and the at least one supplemental fixation element.

In many embodiments, the plate is further configured with a plurality of cannulated barrels that extend distally and the plurality of cannulated barrels are configured to receive the primary fixation element and the at least one supplemental fixation element.

In many embodiments, the bone is a calcar of a femur.

In many embodiments, the device is further configured for affixation in a pertrochanteric region.

In many embodiments, the primary fixation element is configured to terminate within a distance to an end of the bone.

In many embodiments, the distance is 10 mm.

In many embodiments, the primary fixation element is configured to prevent rotation.

In many embodiments, the primary fixation element comprises a channel to prevent rotation.

In many embodiments, the cannulated barrel configured to receive the primary fixation device, is further configured with a geometry to prevent rotation.

In many embodiments, the cannulated barrel configured to receive the primary fixation device is further configured to be angulated and positioned proximal to a calcar.

In many embodiments, the cannulated barrel configured to receive the primary fixation device is further configured to prevent caudal displacement of a femoral head and femoral neck after insertion of the primary fixation device.

In many embodiments, at least one of: the supplemental fixation element, and the positioning fixation element, comprises a locking or nonlocking bone screw.

In many embodiments, the bone screw is a cancellous bone screw.

In many embodiments, the cancellous bone screw is cannulated.

In many embodiments, the cannulated barrel configured to receive the supplemental fixation device is further configured to be angulated so that the supplemental fixation device is positioned for fixation across the fracture.

In many embodiments, the plate is malleable.

In many embodiments, the plate is malleable and configured for angulation of the plurality of barrels.

In many embodiments, the tapered end of the plate is further configured to be contoured to match the bone.

In many embodiments, the device is further configured so that the fracture cannot compress more than a set distance once the device is affixed.

In many embodiments, the set distance is 5 mm.

In many embodiments, the device further comprises a mechanism configured to prevent femoral neck shortening.

In many embodiments, the primary fixation device is hollow.

In many embodiments, the primary fixation device is configured to couple to a jig for alignment and insertion.

In many embodiments, the device further comprises a buttress clip with a plurality of holes configured to receive the positioning fixation element or an additional fixation element and configured to resist rotational angulation and translation.

In many embodiments, the buttress clip is further configured for insertion under musculature of a femur, over an anterior cortex of the femur and applied to a caudal aspect of a femoral neck.

In many embodiments, the buttress clip is further configured for insertion without exposure of the fracture site.

In many embodiments, the buttress clip is further configured so that at least one hole of the buttress clip and at least one hole of the plate configured to receive the positioning fixation element align such that the positioning fixation element affixes both the buttress clip and the plate.

In many embodiments, the buttress clip is further configured so that no more than two positioning and additional fixation element axes are within a plane.

Many embodiments of the disclosure are directed to, a jig for affixation of a medical implant comprises, at least one armature, at least one guide element, and at least one alignment hole; wherein the at least one armature is configured to couple to the medical implant and the medical implant comprises a plate that tapers at an end and the medical implant is configured for fixation of a bone fracture, to conform to a bone, to receive a primary fixation element, and to receive at least one supplemental fixation element; wherein the tapered end of the plate is configured with at least one hole to receive at least one positioning fixation element; wherein the at least one guide element is configured to couple to at least one bone fragment; wherein the jig is configured to rotate and translate the bone fragment relative to the bone; wherein rotation and translation of the jig aligns at least one end of the jig with a point on the bone; and wherein the at least one alignment hole is configured to align a surgical tool with the point on the bone.

In many embodiments, the medical implant further comprises a plurality of cannulated barrels that extend distally from the plate, and the cannulated barrels are configured to receive the primary fixation element and at least one supplemental fixation element; and the jig is further configured to align the surgical tool to prepare holes in the bone to accommodate the cannulated barrel.

In many embodiments, the medical implant further comprises a buttress clip with a plurality of holes configured to receive a positioning fixation element or an additional fixation element and the buttress clip is configured to resist rotational angulation and translation; wherein the jig is further configured to align the surgical tool to prepare holes in the bone for the fixation of the positioning fixation element and the additional fixation element; and wherein the jig is further configured to align the positioning fixation element and the additional fixation element with a hole axis.

Many embodiments of the disclosure are directed to a method of fracture fixation comprising aligning a plate with a bone for fracture fixation; preparing holes in the bone for a plurality of fixation elements; conforming the plate to the bone for fixation; aligning a primary fixation element with a primary fixation axis; inserting a primary fixation element through the plate and into the bone; aligning a supplemental fixation element with a supplemental fixation axis; inserting a supplemental fixation element through the plate and into the bone; applying compression force across a fracture with at least one supplemental fixation element; aligning and inserting at least one positioning fixation element; and securing the plate to the bone with the at least one positioning fixation element.

In many embodiments, the method further comprises positioning a jig to align a surgical tool with at least one point on the bone and preparing holes in the bone with the surgical tool in the bone at the at least one point for the fixation elements.

In many embodiments, the method further comprises affixing a buttress clip configured to resist rotational angulation and translation.

In many embodiments, the primary fixation element is configured to function as an intermediary buttress to resist rotation, angulation, and shear and for insertion without angulation or rotation.

In many embodiments, the method further comprises, coupling a guide element to a first portion of a bone along a first axis on a first side of the fracture; positioning the jig to align a first jig hole with the first axis; translating the jig proximal to the bone so the guide element passes through the first jig hole; positioning the jig to align a second jig hole with a second portion of the bone along a second axis on the second side of the fracture; preparing at least one hole in the second portion of the bone through the second jig hole; translating the at least one guide element through the second jig hole and coupling the second at least one guide element to the second portion of the bone; translating the jig to align the first side of the fracture and the second side of the fracture; rotating the jig to align the first side of the fracture and the second side of the fracture; aligning a plurality of fixation device holes with the bone; preparing a plurality of fixation device holes into the first portion of the bone and the second portion of the bone through the plurality of fixation device holes in the jig; securing the fixation device to the bone.

In many embodiments, the method further comprises, inserting a buttress clip through a passageway in the jig; positioning the buttress clip on the bone; coupling a buttress clip fixation element to the bone; securing the buttress clip to the bone.

In many embodiments, the method further comprises, rotating a compression nut to translate the jig relative to the bone.

In many embodiments, the method further comprises, aligning a buttress clip fixation guide element with the buttress clip; passing the buttress clip fixation element through the buttress clip fixation element guide.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as embodiments of the invention and should not be construed as a complete recitation of the scope of the invention, wherein:

FIG. 1 depicts a flow chart for a method of implanting a fracture fixation device according to the disclosure.

FIG. 2A illustrates an exemplary fracture fixation device in accordance with some embodiments.

FIG. 2B illustrates a detailed view of the plate tail and fixation elements in accordance with some embodiments.

FIGS. 2C and 2D illustrate an exemplary fracture fixation device with cannulated barrels in accordance with some embodiments.

FIGS. 3A and 3B illustrate an exemplary fracture fixation device and buttress clip in accordance with some embodiments.

FIG. 4 illustrates a locking mechanism in accordance with some embodiments.

FIG. 5 illustrates a locking mechanism in accordance with some embodiments.

FIGS. 6A and 6B illustrate a locking mechanism in accordance with some embodiments.

FIG. 7 illustrates a flow chart for utilizing a jig to facilitate fracture fixation in accordance with some embodiments.

FIG. 8 illustrates a schematic of a bone fixation jig in accordance with some embodiments.

FIG. 9 illustrates a schematic of a jig armature in accordance with some embodiments.

FIGS. 10A through 10C illustrate fracture fixation with a jig in accordance with some embodiments.

FIG. 11 illustrates a side view of a fixation jig in accordance with some embodiments.

### DETAILED DESCRIPTION OF THE INVENTION

Various embodiments are directed toward systems and devices for fixed-angle fixation of fractures in the femoral neck and methods of their use. In accordance with many embodiments, an operating surgeon can fixate fractures in the femoral neck without requiring an open reduction technique or exposure. In various embodiments, the devices provide suitable implant spread, calcar support, and medial femoral neck buttress support so that the operating surgeon can perform a reduction with minimal assistance. In many cases, technical errors can be prevented if there is sufficient implant spread, calcar support, and medial femoral neck buttress support such as the provided by numerous embodiments. Technical errors can lead to implant failure and impaired fracture healing. Additionally, numerous embodiments provide a mechanism that restricts the shortening of the femoral neck. The shortening of the femoral neck can impair a patient's functional outcomes, such as their ability to walk and quality of life.

In many embodiments, the fixation device comprises a plate, a primary fixation element, at least one supplemental fixation element, and at least one positioning element, which creates three linked fixed-angle fracture fixation components. Various embodiments also comprises a buttress clip and additional fixation elements that provide for a fourth fixed-angle component for fracture fixation. Fixed-angle fracture fixation devices are associated with a lower risk of fixation and implant failure in patients of all ages with a fracture of the femoral neck.

Fractures in the femoral neck are a common form of hip fracture, often requiring surgical intervention to promote bone healing and restore functionality. Typically, these types of fractures are treated with a bone fixation system that involves the insertion of an implant shaft through a bone plate. This procedure requires open-reduction surgery, where the affected area is surgically opened to realign the bones and fix them in place with screws, plates, or rods. However, such procedures can be invasive, with high associated risks of complications, including infection, extended recovery times, and implant failure. Various embodiments of the disclosure overcome these challenges by enabling the surgeon to perform a closed reduction. A closed reduction involves manipulating the bones back into their proper position without exposure. A closed reduction reduces the risk of nonunion and implant failure and, consequently, the need for patients to undergo a subsequent operation as a result of such complications.

The bone fixation systems presented in this disclosure are designed to be compatible with closed reduction of the fracture by the operating surgeon without additional assistants, thus minimizing the need for open-reduction surgery, reducing the risk of complications, and allowing for a quicker recovery time for the patient. As such, the bone fixation systems presented in this disclosure are a promising solution for patients who require surgical intervention for femoral neck fractures and can significantly improve their quality of life.

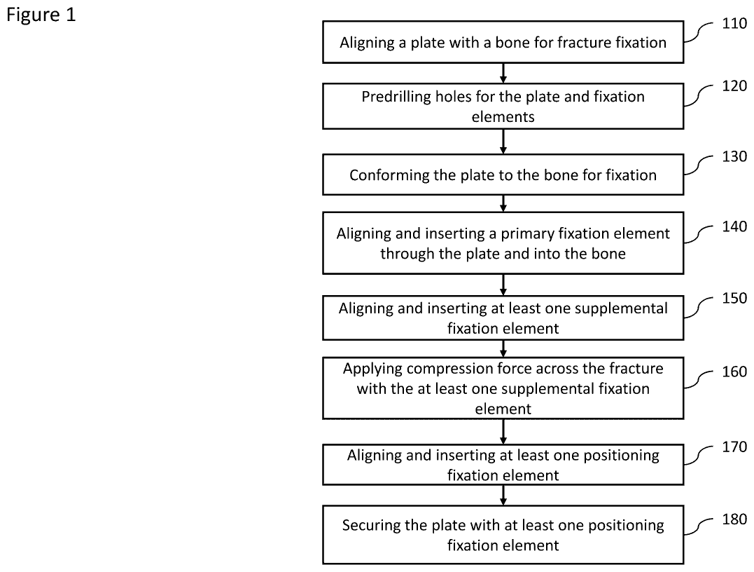

FIG. 1 depicts a flow chart for a method of implanting a fracture fixation device in accordance with numerous embodiments. At step 110, a plate is oriented by an operating surgeon, aligning the plate with fractured bone parts for fracture fixation. At step 120, the surgeon prepares holes in the bone to accommodate the plate's features, such as its barrel configuration, and for the fixation elements, such as the bone screws. Often the surgeon may opt to utilize a jig to facilitate alignment and preparation of the bone for placement of the fixation elements, such as drilling holes in the bone. At step 130, the surgeon conforms the plate to the bone for fixation. Then at step 140, the surgeon aligns the primary fixation element with the holes prepared in the bone parts and with the plate and then inserts the primary fixation element through the plate and into the bone. At step 150, the surgeon similarly aligns and inserts a supplemental fixation element. Once aligned at step 160, the surgeon applies a compression force across the fracture with the supplemental fixation element, often in the form of a cancellous bone screw. Then at step 170, the surgeon aligns and inserts a positioning fixation element. At step 180, the surgeon further secures the plate to the bone with a positioning fixation element. In some instances, the surgeon may opt to utilize a buttress clip to supplement the fixation, in such cases the position fixation element can be utilized to secure both the buttress clip and the plate.

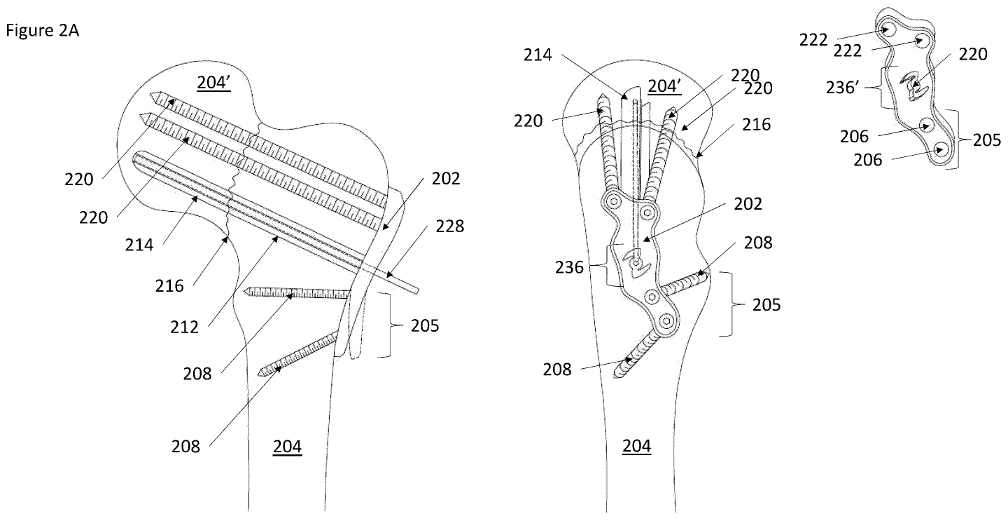

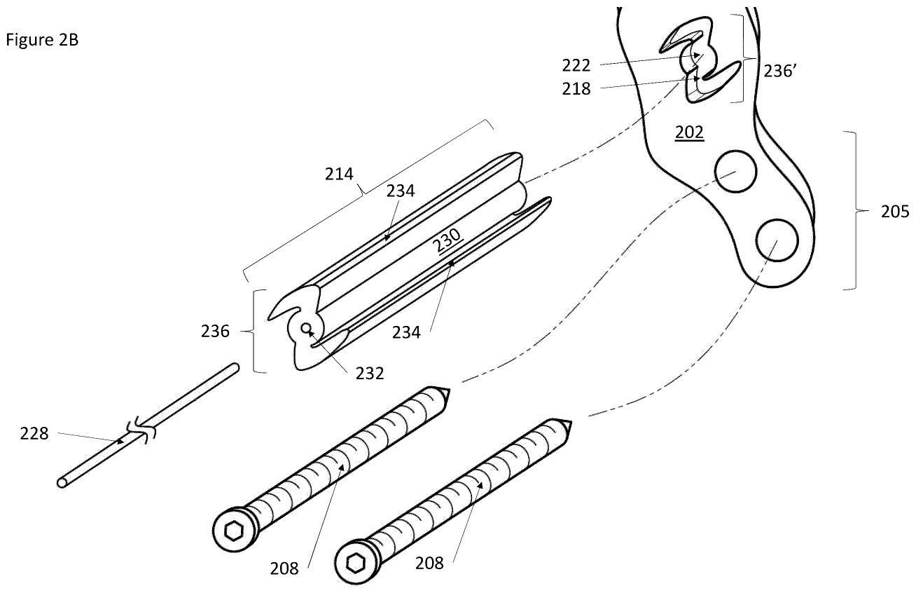

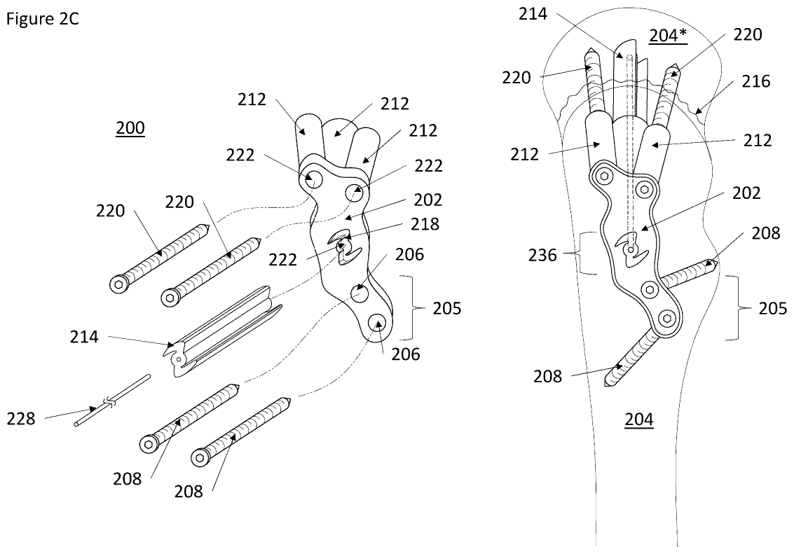

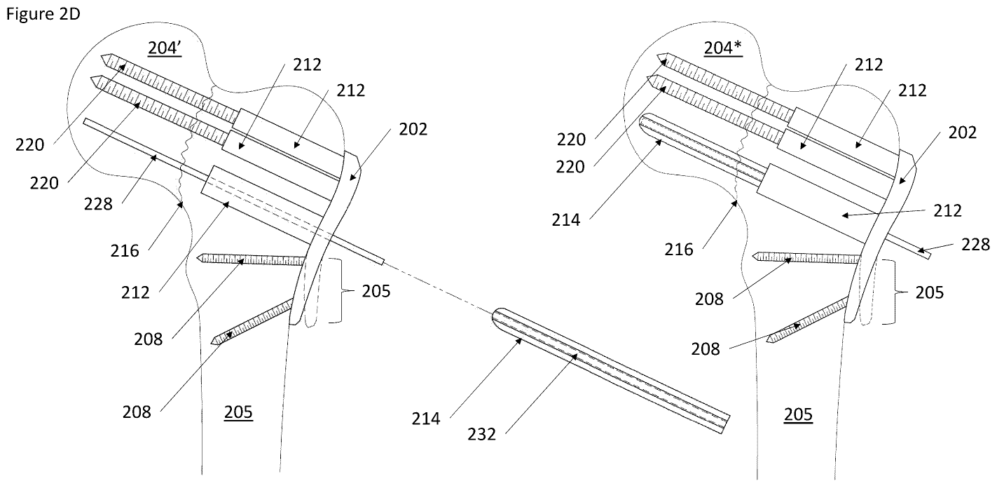

FIGS. 2A to 2D depict a fracture fixation device 200 in accordance with many embodiments, as will be described in greater detail in the section below.

### The Plate

In many embodiments and as illustrated in FIGS. 2A and 2B, a plate 202 is affixed to the proximal femur 204 in the pertrochanteric region using a jig (not shown). In some embodiments, the jig is utilized for preparing holes in the bone. In many such embodiments, the prepared holes are configured for the insertion of the fixation elements. In many embodiments, the jig is configured for the alignment of surgical tools such as tools for drilling holes in the bone 204.

In numerous embodiments, the plate 202 tapers at one end into a tail 205 which contains a plurality of holes 206 holes through which a positioning fixation element can be employed to affix the plate 208 to the bone 204. In many embodiments, the plate 202 is malleable, and the tail 205 can be positioned by the surgeon to match the morphology of bone 204, such as the lateral cortex of the femur before or during insertion.

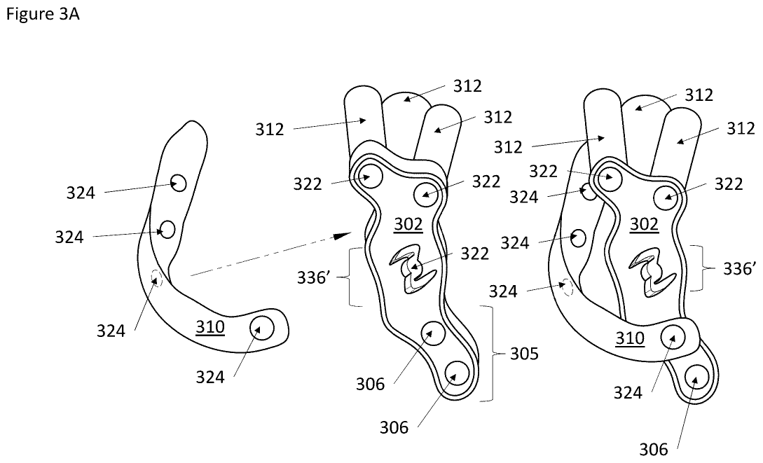

In some embodiments and as illustrated in FIGS. 3A and 3B, the tail section 305 includes a mating mechanism for secure placement of the buttress clip 310. In many such embodiments, a positioning fixation element 308, such as a bone screw, is inserted to secure both the plate 302 and the buttress clip 310 to one another and to the bone 304 (femoral shaft).

In many embodiments, the plate 202 is configured with a plurality of boreholes 222. In many such embodiments, the primary fixation element 214 is inserted through one of the boreholes 222. In many such embodiments, the borehole 222 through which the primary fixation element 214 is inserted is of a larger diameter and is the more central borehole. In numerous embodiments, the borehole is slotted 218, matching the outer morphology of the primary fixation element 214, such that the primary fixation element 214 may translate freely along the axis of the borehole 222 without angulation or rotation.

In many embodiments, the supplemental fixation element 220 is inserted through one of the boreholes 222. In many such embodiments, the borehole through which the supplemental fixation device 220 is inserted is of a small diameter and is the more proximal barrel. In many embodiments, the borehole 222 configured to receive a nonlocking supplemental fixation element 220 is further configured to match the head configuration of the supplemental fixation element 220 so that the head can be compressed against the borehole 222. In some embodiments, the plate 202 or borehole 222 is configured with a locking mechanism to retain the supplemental fixation element 220.

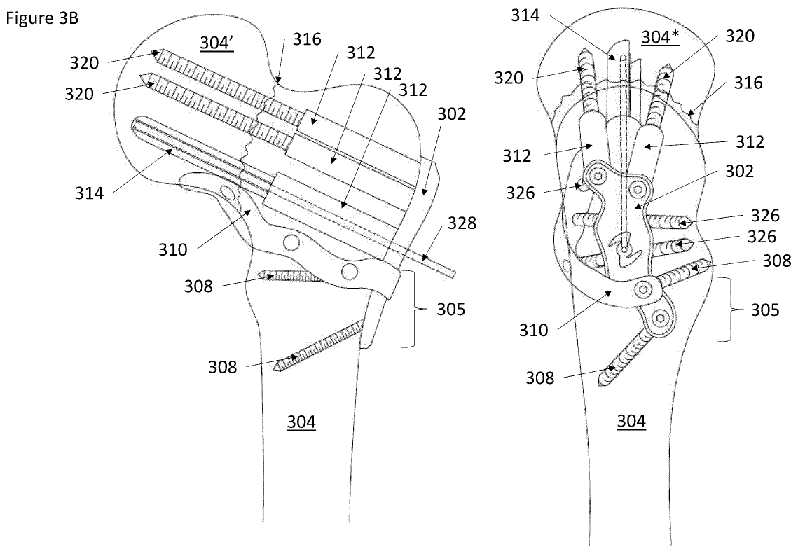

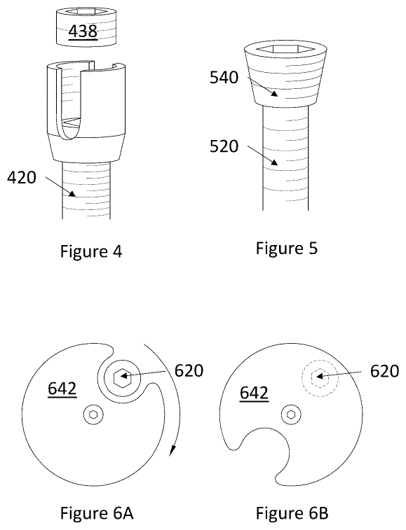

FIGS. 4, 5, 6A, and 6B illustrate various exemplary locking mechanisms. FIG. 4 illustrates an exemplary locking insert 438 that contains and secures the supplemental fixation element 420 after insertion. FIG. 5 illustrates an exemplary locking cancellous bone screw with a threaded countersink 540 that mates with a complementary locking thread in the borehole 222. FIGS. 6A and 6B illustrate a rotating cap 642 configured to receive the supplemental fixation element 620 (FIG. 6A) and rotate after the supplemental fixation element 620 (FIG. 6B) is inserted to secure and retain the supplemental fixation element 620. Numerous other locking mechanisms that would be known to one skilled in the art could also be utilized to secure the supplemental fixation element 220 after installation.

In many embodiments, such as those illustrated in FIGS. 2C and 2D and FIGS. 3A and 3B, the plate contains a plurality of cannulated barrels 212. In many such embodiments, the primary fixation element 214 is inserted through one of the cannulated barrels 212. In many such embodiments, the cannulated barrel 212 through which the primary fixation device 214 is inserted is of a larger diameter and is the more central barrel. In various embodiments, the barrel 212 and the primary fixation element 214 are configured so that either rests along the calcar bone of the proximal femur 204 to prevent caudal displacement of the femoral head and neck fragment 204′ after the primary fixation element 214 is implanted across the fracture 216. In numerous embodiments, the bore of the barrel is slotted 218, matching the outer morphology of the primary fixation element 214, such that the primary fixation element 214 may translate freely along the axis of the barrel 212 and femoral neck without angulation or rotation.

In many embodiments, the supplemental fixation element 220 is inserted through one of the cannulated barrels 212. In many such embodiments, the barrels 212 through which the supplemental fixation elements 220 are inserted can be angulated by the operating surgeon such that their orientation is optimized for fixation across the femoral neck fracture 216. In many embodiments, the cannulated barrel 212 through which the supplemental fixation device 220 is inserted is of a small diameter and is the more proximal barrel.

In numerous embodiments and illustrated in FIGS. 3A and 3B, the boreholes, barrels 312 and holes 306 of the plate 302 and the holes 324 of the buttress clip 310 are configured so that no more than two perforations in the bone 304 or two fixation elements (primary 314, supplemental 320, positioning 308, and additional 326), are within the same plane to reduce stress concentration and minimize the risk of implant failures and fractures.

### Fixation Elements

In many embodiments, the fixation elements (primary 314, supplemental 320, positioning 308, and additional 326) secure the plate 302 or buttress clip 310 to the bone 304, creating a fixed-angle device. In many such embodiments, the fixation elements are bone screws or cancellous bone screws. In many such embodiments, the cancellous bone screws provide supplemental fixation, securing the femoral neck and head 304′ to the remainder of the femur 304 across the fracture 316 and the plate 302 and buttress clip 310 to the bone 304. In many embodiments, the bone screws, once affixed, resist translation such that the fracture 316 may not compress beyond a maximal distance. In some such embodiments, the maximal distance is less than 5 mm. In other embodiments, the maximal distance is 5 mm or more. In many such embodiments, the compression limiting mechanism prevents femoral neck shortening of 5 mm or more. Neck shortening can impair a patient's functional outcome, such as their ability to walk and quality of life.

In many embodiments, the cancellous bone screws are of two varieties: locking and nonlocking. In many such embodiments, nonlocking compression cancellous bone screws are often inserted first to compress across the fracture 316. In some such embodiments, the nonlocking bone screw is configured with variable pitch threads to compress across the fracture site. In many embodiments locking cancellous bone screws are then inserted to secure the reduction. In many such embodiments, the locking bone screw is configured with non-variable pitch threads to secure the reduction and prevent pull-out of the fixation. In various embodiments, either a nonlocking compression bone screw or a locking fixation bone screw may be inserted into the barrels 312 of the plate 302, the holes 306 of the tail 305, or the holes 324 of the buttress clip 310.

In numerous embodiments, the fixation elements (primary 314, supplemental 320, positioning 308, and additional 326) are configured to permit insertion over a guide pin or wire 328, for example, a cannulated bone screw, and assist the operating surgeon in securing the device in the optimal orientation and achieve a widespread fixation within the femoral head and neck and accommodate the variations in bone morphology between patients. Optimization of the cancellous bone screw spread within the femoral head and neck is associated with superior biomechanics of fracture fixation and lower risk of fixation failure. In many embodiments, the fixation elements (e.g., bone screws) are configured for fixation within 10 mm of the cartilage surface of the femoral head and hip joint.

### The Primary Fixation Element

In many embodiments and as illustrated in FIG. 2B, the primary fixation element 214 has an elongated implant shaft 230 with a channel or hollow center 232 extending along its length. In some embodiments, the implant shaft 230 is configured to mate to the borehole 222 and barrel 212 of the plate 302. In many such embodiments, the hollow or channel 232 is configured to receive a guide wire 228 to assist the surgeon with insertion and, if necessary, removal of the element. In many embodiments, the primary fixation element also contains threads for the attachments of a mechanism for insertion and removal. In numerous embodiments, the outer walls 234 of the implant shaft 230 are configured to displace and compress adjacent bone during insertion.

In various embodiments, the outer walls of the implant shaft 234 are configured with a bone-engaging features. In some such embodiments, the bone-engaging feature is a smooth surface, a screw thread, or a fluted surface.

In many embodiments, the outer wall 234, and the hollow or channel 232 are configured to form multi-faceted surfaces that prevent translation of the bone fixation device 200 relative to the bone 204 and the fracture 216. In accordance with many embodiments, the outer walls of the implant shaft 234 are configured to prevent rotation relative to the bone plate 202, through which the primary fixation element 214 is inserted. In various embodiments, the outer walls 234 of the shaft 230 are configured to include at least one geometric shape 236 arranged to mate with a complementary shape 236′ on the plate 202. In many such embodiments, the position of the geometric shape 236 determines the orientation of the implant shaft 230 when inserted through the bone plate 202. In some embodiments, geometric shape 236 will consist of a plurality of geometric structures, such as triangular notches, symmetrically aligned along the shaft 230, such as on the opposing sides of the implant shaft 230. In other embodiments, a single structure extends along an outer wall of the shaft 230.

In numerous embodiments, the primary fixation element 214 is the primary form of fixation securing the plate 202 to the bone 204 and the femoral neck and head 204′ to the remainder of the femur 204 across the fracture 216. In numerous embodiments, the tip of the primary fixation element 214 is contoured to fit within the femoral head and neck 204′ and as deep within the bone and as close to the cartilage surface of the femoral head as possible, to maximize the tip-apex distance of the implant.

In many embodiments, the primary fixation device 214 does not rotate during insertion. Rotation of the fracture during insertion of a bone screw is a known cause of loss of reduction and is considered a technical error in the treatment of femoral neck fractures with bone screws and sliding hip screw devices.

In many embodiments, the primary fixation element 214 is positioned such that it resets against the calcar medial cortex of the intact femur, such that the primary fixation element functions as an intermediary buttress to resist rotational angulation and vertical shear translation. Rotational angulation and vertical shear translation are the primary mechanisms of failure for femoral neck fixation, and the malpositioning of screws and or blades has often been associated with the failure of other devices.

### The Buttress Clip

Numerous embodiments, such as the exemplary embodiment illustrated in FIGS. 3A and 3B, further contain a buttress clip 310 that is configured as a curved support armature that resists rotational angulation and vertical shear translation about the femoral neck fracture 316. In many such embodiments, the buttress clip 310 provides supplemental fixation, biomechanical support, and resistance to fixation failure and lowers the risk of nonunion and the need for reoperation. In many embodiments, the buttress clip 310 is inserted under the musculature of the anterior and lateral femur, curves over the anterior cortex of the femur, and is applied to the caudal aspect of the femoral neck medial to the fracture. Unlike many traditional fixation devices, which require an open approach, the buttress clip 310, in accordance with various embodiments, can be applied without open exposure of the fracture site, which preserves the blood supply to the femoral neck. In numerous embodiments, the buttress clip 310 mates to the tail 305 of the plate 302. In many such embodiments, the buttress clip 310 is secured to the plate 302 with a positioning fixation element 308, such as a bone screw. In numerous embodiments, the joining of the buttress clip 310 and the plate 302 creates a fourth fixed angle, further securing the (femoral neck) fracture 316. In other embodiments, the buttress clip 310 is secured to the bone 304 (proximal femur) separately from the plate 302 with an additional fixation element 326, such as a bone screw. In many such embodiments, the bone screw compresses the buttress clip 310 to the bone 304. In numerous such embodiments, the compression results in preloading the supportive cranial force and applies a force to the caudal aspect of the femoral neck medial to the femoral neck fracture.

### The Installation Jig

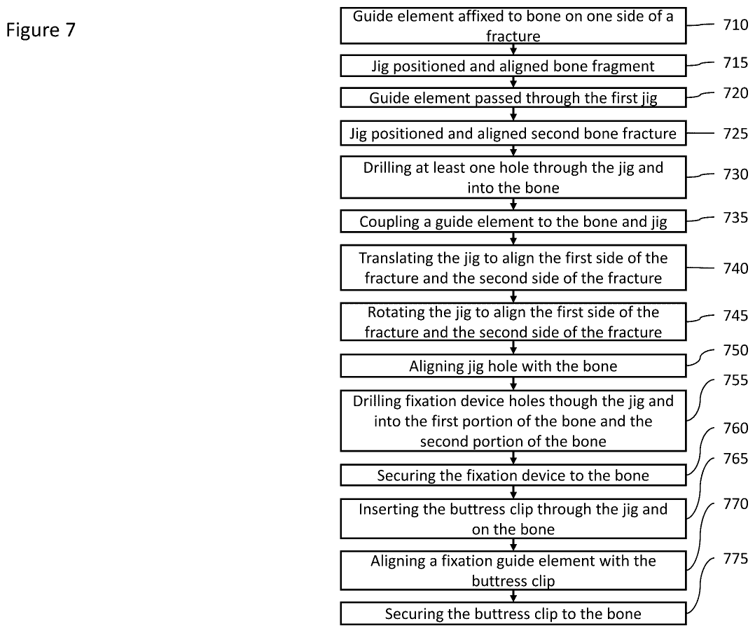

FIG. 7 depicts a flow chart for a method of utilizing a jig to facilitate alignment, preparing the bone, and implanting a fracture fixation device in accordance with numerous embodiments. At step 710, a central guide element such as a guide wire or guide pin is installed and secured into a first portion of a bone on the first side of a fracture. At step 715, the jig is positioned, aligning a positioning hole in the jig with the axis of the central guide element. At step 720, the jig is moved toward the bone, and the guide wire is passed through the positioning hole in the jig. In some embodiments, a compression nut is secured to the central guide element on the end of the guide element distal to the bone to secure the jig and enable the application of a compression force. At step 725, the jig is aligned with a second portion of the bone on the second side of the fracture. At step 730, the bone is prepared with at least one fixation hole made into the second portion of the bone, such as by drilling into the bone. In many embodiments the fixation hole is located by inserting a bone preparation mechanism, such as a drill bit through an alignment hole in the jig. At step 735, at least one guide pin is passed through a corresponding alignment hole in the jig and secured to the bone in the prepared hole. At step 740, the jig is translated to align the first side of the fracture and the second side of the fracture. In some embodiments, compression and distraction, nuts are installed on the central guide element and the guide pin. In many such embodiments, the nuts are used to apply compression and distraction forces that translate the jig relative to the bone. At step 745, the jig is rotated to align the first side of the fracture and the second side of the fracture. In some embodiments, the jig comprises a ratchet mechanism used to manipulate and secure the alignment of the jig. At step 750, the jig is positioned to guide the preparation of the bone with a plurality of fixation device holes for fixation. At step 755, the plurality of fixation device holes are made into the aligned first and second portions of the bone through holes in the jig. At step 760, the fixation device is secured to the bone. In many embodiments, a compression force is applied by the compression nut on the central guide element to facilitate implanting the fixation device. In some embodiments at step 765, a buttress clip is inserted through a passageway in the jig so that it can be positioned on the bone relative to the fixation device without needing to remove the jig. At step 770, a buttress clip fixation guide is aligned with the buttress clip. At step 775, a buttress clip fixation element is passed through the buttress clip fixation guide and coupled to the bone, securing the buttress clip to the bone.

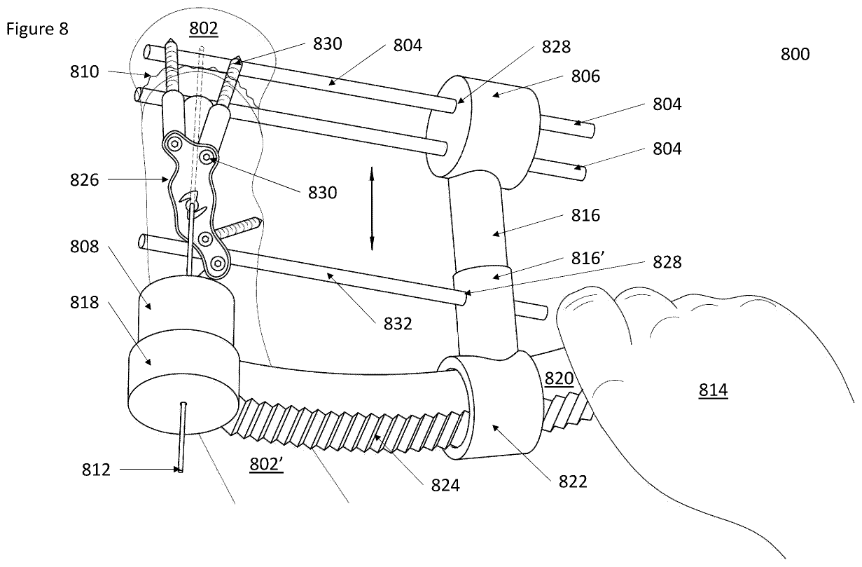

FIGS. 8 through 11 depict a jig utilizing a jig to facilitate alignment, bone preparation, and implanting a fracture fixation device in accordance with numerous embodiments, as will be described in greater detail in the section below.

FIG. 8 depicts a schematic of a fractured bone, bone fixation, and installation jig 800 in accordance with many embodiments. In many embodiments, the jig 800 comprises mechanisms for positioning, aligning, securing, and inserting surgical tools, fixation elements, and fixation devices for the reduction and fixation of a fractured bone 802 and 802′, such as the proximal femur. In many embodiments, the jig 800 is configured to attach to the lateral side of a proximal femur fracture fragment and a femoral neck fracture, such as those involving the peritrochanteric region of the femur and the shaft. In many such embodiments, the jig 800 is coupled with the fractured bone 802 with at least one guide element 804. In some embodiments, the jig 800 is configured for fixation of the proximal femur medial to the femoral neck fracture, such as a fracture involving the femoral neck 802′ and head 802. In many embodiments, the jig 800 comprises a plurality of armatures 806 and 808 that are configured to couple with each of the fracture fragments, such as head 802 and neck 802′ lateral fracture fragments. In many such embodiments, at least one guide element 804 couples a first armature 806 with a first portion of bone 802 on a first side of a fracture 810, and a second guide element 812 couples a second armature 808 with a second portion of bone 802′ on a second side of the fracture 810. In many embodiments, the armatures 806 and 808 are configured so that when coupled to the bone fragments 802 and 802′, an operator 814, such as a surgeon, can manipulate the bone fragments 802 and 802′ with the jig 800 in a controlled and stable manner. In many embodiments, the jig 800 enables a single operator 814 to manipulate and position the bone fragments 802 and 802′ for fixation without assistance, such as from an assistant surgeon.

In many embodiments, the jig 800 comprises articulation elements 816 and 816′ configured to guide placement of at least one guide element, such as a guide pin 804 or wire 812 into the femoral head 802 and neck 802′. In some embodiments, the jig 800 further comprises at least one nut 818 that is coupled to a guide element 804 and 812 and configured to apply a force such as a compression or distraction force on the jig 800. In many such embodiments, the nut 818 translates along the fixation guide element 804 and 812. In many such embodiments, rotation of the nut 818 applies a force that is configured to translate the jig 800 relative to bone fragments 802 and 802′. In many such embodiments, the articulation elements 816 are configured to articulate when the jig translates.

In some embodiments, the jig 800 comprises a grip element 820 configured to facilitate manipulation of the jig. In some embodiments, the jig 800 further comprises a ratchet mechanism 822 configured to manipulate and secure the alignment of the jig armature elements 806 and 808 relative to the bone fragments 802 and 802′. In many such embodiments, the jig is further configured with a geometry complementary to the ratchet mechanism 824. In many such embodiments, the portion of the jig with the geometry commentary to the ratchet 824 is configured to pass through the ratchet mechanism 822, and the ratchet mechanism is further configured to selectively couple with the jig geometry and limit the movement of the jig and coupled bone fragments relative to each other. In some embodiments, the ratchet mechanism 822 is configured to limit translation. In many embodiments, the ratchet mechanism 822 is configured to limit rotation.

In many embodiments, translation along the guide elements 804 and 812 imparts a force on the jig 800 and/or the fixation device 826, pushing or pulling the jig 800 and/or fixation device 826 into an alignment. In many embodiments, the jig 800 is configured for the insertion of a fracture fixation device 826. In many such embodiments, the jig 800 is further configured for positioning the fixation device 826 against the bone, tendon, and muscle. In many embodiments, the jig 800 further comprises at least one alignment hole 828 configured to orient and align elements necessary for bone fixation, with an axis including but not limited to surgical tools, implant devices 826, fixation elements 830, and guide elements 804 and 812. In some embodiments, the at least one alignment hole 828 is configured to align, position, and receive guide elements 806 and 812, fixation elements 830. In many embodiments, the at least one alignment hole 828 is configured to receive a fixation element guide 832 configured to orient and facilitate installation of fixation elements 830 along an axis. In some embodiments, the least one alignment hole 828 is further configured to receive, position, and align surgical tools such as drilling and cutting elements.

In many embodiments, the jig is configured to allow for the direct placement of fixation elements such as screws. In many embodiments, the jig is configured for placement of the buttress clip without removal of the jig. In many such embodiments, the jig is configured to allow placement of fixation elements for fixation of the buttress clip without removal of the jig. In some embodiments, the jig comprises at least one passageway configured to allow access to the bone fixation site. In some embodiments, the passageways are configured so fixation elements, tools, and devices can be passed through the passageway for fixation.

In some embodiments, the jig is radiolucent. In many such embodiments, the jig is permeable to radiation and X-rays. In many such embodiments, different elements of the jig are configured with different degrees of radiolucency. In many such embodiments, the degree of radiolucency of the elements is configured so their orientation and arrangement can be viewed on an X-ray. In some embodiments, the jig is configured to be sufficiently radiolucent so that features behind the jig can be viewed on a medical image such as an x-ray. In many such embodiments, the fixation implant can be viewed through the jig on a medical image. In numerous such embodiments, the radiolucency is configured such that the orientation of the jig relative to the bone fixation can be viewed through the jig on a medical image such as an X-ray.

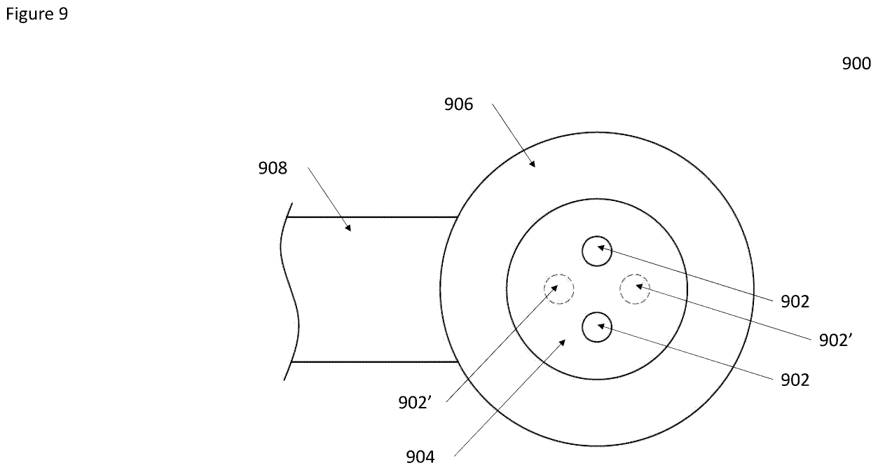

FIG. 9 depicts a schematic of an armature 900 in accordance with several embodiments. In many embodiments, the armature is configured with at least one alignment hole 902. In many such embodiments, the at least one alignment hole 902 is disposed on an inner portion of the armature 904. In many embodiments, the inner portion of the armature 904 is configured to rotate relative to an outer portion of the armature 906. In many embodiments, alignment holes 902 are configured to receive guide elements that couple to bone fragments. In many such embodiments, the armature 900 is further configured so that the rotation of the jig 908 rotates the outer portion of the armature 906 relative to the inner portion of the armature 904. In many such embodiments, the jig is further configured so that jig 908 rotates relative to the bone fragment, but the bone fragment does not rotate relative to the inner portion of the armature 904 and guide elements coupled to the inner portion. In some embodiments, the armature 900 further comprises at least one tab element that is coupled to either the inner 904 or outer portion 906 of the aperture 900 and configured to facilitate rotation of the inner 904 and outer 906 portions relative to each other. In some embodiments, the armature 900 comprises a ratchet mechanism. In many such embodiments, the ratchet mechanism is configured to selectively limit the rotation of the inner 904 and outer 906 portions relative to each other.

In many embodiments, the jig is configured to have multiple degrees of freedom. In many embodiments, the jig is configured for controlled rotation, angulation, and translation of the coupling elements, fixation elements, fixation devices, and bone fragments. In many embodiments, the jig is configured to rotate the fracture fragments on an axial plane. In many embodiments, the jig is configured to rotate the fracture fragments on a coronal plane. In many embodiments, the jig is configured to rotate the fracture fragments on a sagittal plane. In many embodiments, the jig is configured for cranial-caudal translation of the fracture fragments. In many embodiments, the jig is configured for medial-lateral translation of the fracture fragments. In many embodiments, the jig is configured for anterior-posterior translation of the fracture fragments.

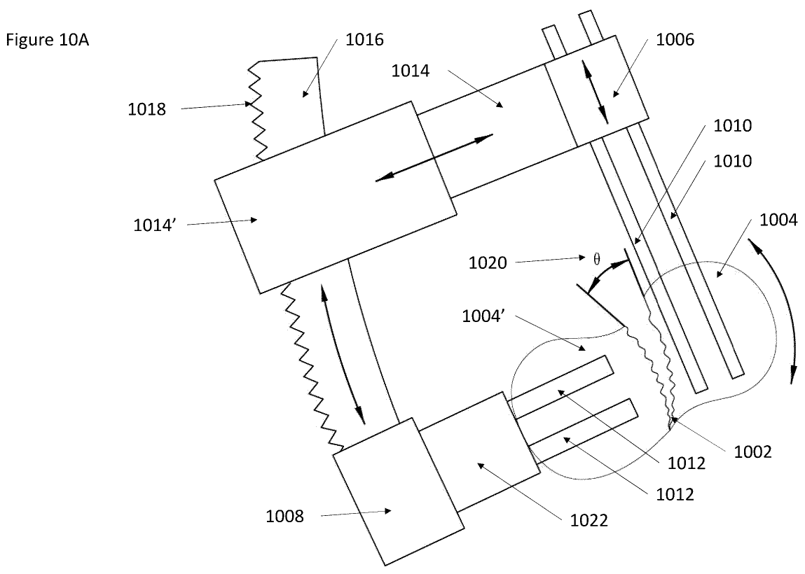

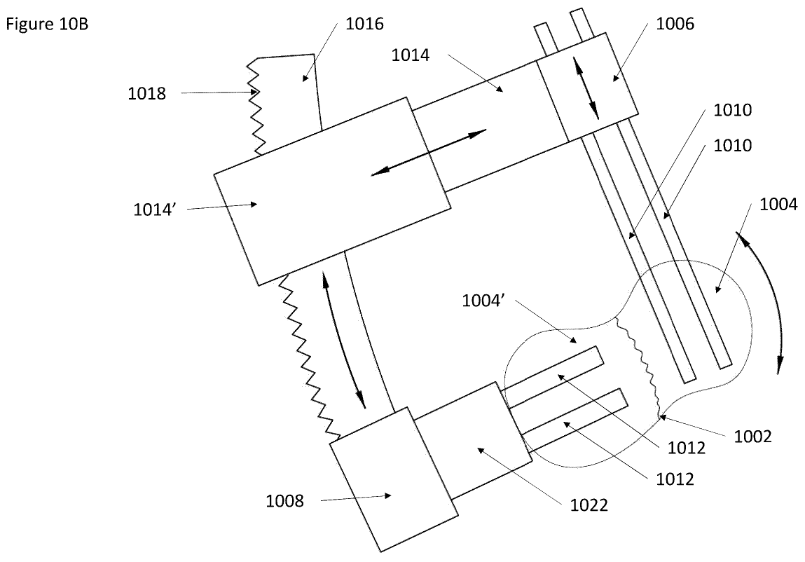

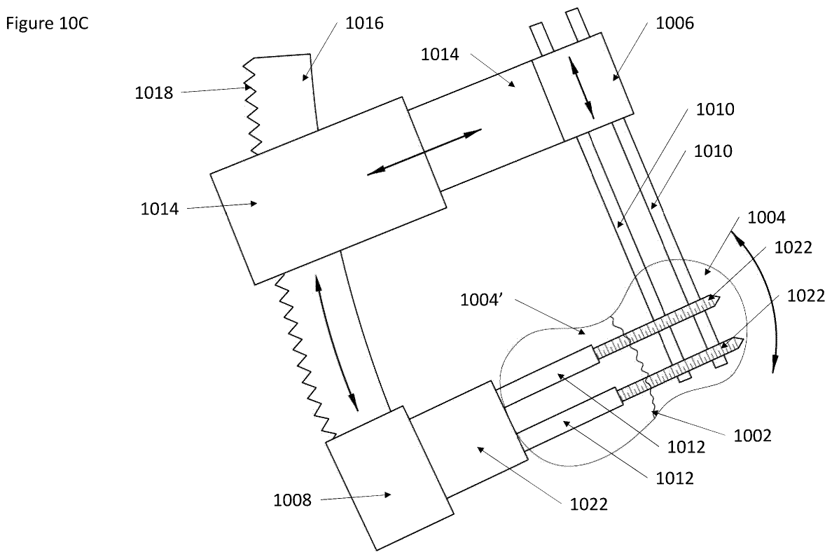

FIGS. 10A through 10C depict the manipulation and fixation of a bone fracture with a jig in accordance with some embodiments. In many embodiments, the jig is configured to reduce a fracture of 1002 without exposure of the fracture site. In many embodiments, the jig is coupled to a bone fragment 1004 on a first side of the fracture 1002 at a first end 1006 and a bone fragment 1004′ on a second side of a bone fracture 1002 at a second end 1008. In many such embodiments, the jig is coupled to the bone fragments 1004 and 1004′ with guide elements 1010 and barrel elements 1012. In many embodiments, the guide elements 1010 and barrel elements 1012 are configured so the jig can manipulate the bone fragments 1004 and 1004′ to align and reduce the fracture 1002. In some embodiments, the barrel elements 1012 are coupled to a bone fixation device 1022. In many embodiments, the guide elements 1010 pass through the first end 1006 of the jig. In many embodiments, the first of the jig end 1006 is configured to translate along the guide elements 1010 and manipulate the position of the first bone fragment 1004 relative to the second bone fragment 1004′. In many embodiments, the first and/or second ends of the jig 1006 and 1008 are disposed on an armature 1014 and 1016. In many embodiments, the armature 1014 is configured to articulate. In many such embodiments, a first portion of the armature 1014 extends from a second portion of the armature 1014′. In many embodiments articulation of the armature 1014 translates the first end of the jig 1006 relative to the second end of the jig 1008. In many such embodiments articulation of the armature 1014 translates the coupled bone fragments and manipulates the position of the first bone fragment 1004 relative to the second bone fragment 1004′.

In many embodiments, the jig comprises a ratchet mechanism, and at least one armature 1016 is configured with a geometry complementary to the ratchet mechanism 1018. In many such embodiments, the armature 1016 is configured to pass through the ratch mechanism. In many embodiments, the ratchet mechanism selectively limits the translation of the armature 1016 relative to the bone fragment 1004. In many embodiments, the ratchet mechanism selectively limits the rotation of the armature 1016 relative to the bone fragment 1004.

In many embodiments, the jig comprises at least two armatures, 1016 and 1014, and each of the at least two armatures couple to different bone fragments, 1004 and 1004′. In many such embodiments, at least two armatures manipulate the bone fragments 1004 and 1004′ independently of each other. In some embodiments, at least one armature is configured with a covered geometry. In many such embodiments, the curved geometry of the armature 1016 is configured as an arc length with a pivot point. In such embodiments, the jig is configured so that translation and articulation of the jig relative to the bone fragments can align the pivot point with the fracture 1002. In many embodiments, translating the curved armature rotates the second bone fragment 1004′ relative to the first bone fragment 1004. In many such embodiments, translating the curved armature 1016 along a curved pathway changes an angle 1020 between the first 1004 and second 1004′ bone fragments.

In many embodiments, the jig can manipulate the first bone fragment 1004 and the second bone fragment 1004′ relative to each other by translating and rotating, moving the fragments relative to each other to reduce the fracture 1002. In many embodiments, the jig can manipulate the bone fragments 1004 and 1004′ with six degrees of freedom. In many embodiments, the jig is configured to manipulate the bone fragments 1004 and 1004′ relative to each other with translational movement along three perpendicular axes. In many embodiments, the jig is configured to move a bone fragment 1004 longitudinally (forward and backward). In many embodiments, the jig is configured to move a bone fragment 1004 laterally (side to side). In many embodiments, the jig is configured to move a bone vertically (up and down). In many embodiments, the jig is configured for yaw rotation (rotation around a vertical axis) of a bone fragment 1004. In many embodiments, the jig is configured for pitch rotation (rotation around a lateral axis) of a bone fragment 1004. In many embodiments, the jig is configured for roll rotation (rotation around a longitudinal axis) of a bone fragment 1004. In many embodiments, the jig is configured to selectively lock movement in each of the degrees of freedom.

In many embodiments, once the bone fragments 1004 and 1004′ have been manipulated to reduce the fracture 1002, all of the jig degree of freedom movements are locked to secure the relative position of the jig and the bone fragments. In many embodiments, once the fracture 1002 has been reduced, at least one fixation element 1022 is installed. In many embodiments, the fixation element 1022 is installed across the fracture 1002. In many embodiments, the jig is configured with at least one passageway through which a fixation element 1022 can pass. In many such embodiments, the passageway is configured so the fixation element 1022 can be installed into the bone fragment 1004 without the removal or repositioning of the jig. In many such embodiments, the passageway is configured to align the fixation element 1022 relative to the fracture 1002.

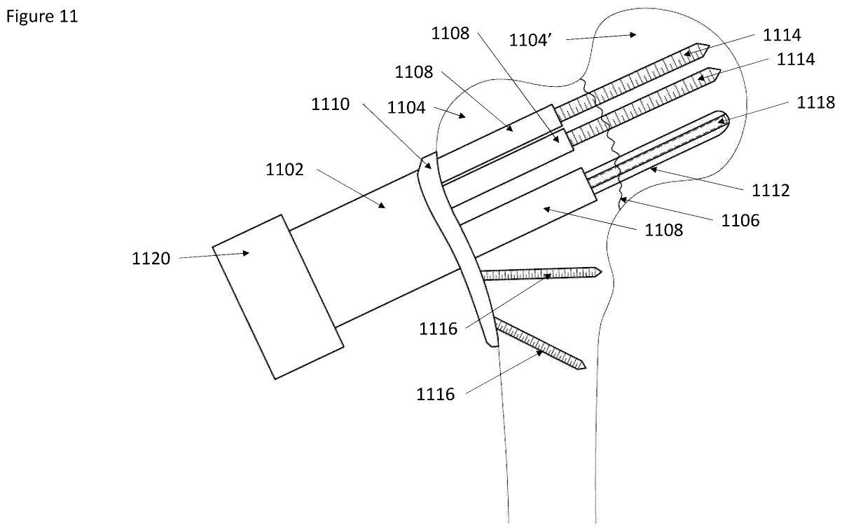

FIG. 11 depicts a side view of the fixation jig and fixation device in accordance with several embodiments. In many embodiments, the jig 1102 is configured to manipulate bone fragments 1104 and 1004′ into alignment to reduce a fracture 1106. The jig 1102 is configured to position tools to prepare holes in the bone to accommodate the barrels 1108 of the fixation device 1110. In many embodiments, the jig 1102 is configured to position tools to prepare holes in the bone through the first bone fragment 1104 and into the second bone fragment 1104′ for a primary fixation element 1112 and at least one secondary fixation element 1114 fixation element. In many embodiments, the jig is configured to position tools to prepare holes in the bone for positioning fixation elements 1116. In many embodiments, the jig 11102 is configured to position a fixation device 1110. In many embodiments, the jig 1102 is configured to install the fixation device 1102 into the prepared holes. In some embodiments, a hallow primary fixation element 1112 is installed over a guide element 1118, such as a guide wire securing the fixation device 1110 to the bone 1104. In many embodiments, the jig 1102 comprises a nut 1120 configured to apply a compression force across the jig 1102, fixation device 1110, and fracture 1106. In many embodiments, the guide element 1118 is threaded, and the nut 1120 comprises complementary threads and is configured so rotation of the nut 1120 applies the compression force. In many embodiments, the compression force seats the fixation device 1110 against the bone 1104. In many embodiments, at least one secondary fixation element 1114 is installed across the fracture 1106, securing the reduction and the fixation device 1110. In some embodiments, positioning fixation elements 1116 are installed to secure the fixation device 1110 to the bone 1104.

### DOCTRINE OF EQUIVALENTS

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the terms “approximately” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

1. A bone fracture fixation device comprising:
a plate, a primary fixation element, at least one supplemental fixation element, and at least one positioning fixation element;
wherein the plate tappers at an end and is further configured with at least one hole to receive the at least one positioning fixation element; and
wherein the plate is configured to conform to a bone for fixation, for insertion without open exposure of a fracture site, and to receive the primary fixation element and the at least one supplemental fixation element.

2. The device of claim 1, wherein the plate is further configured with a plurality of cannulated barrels that extend distally and the plurality of cannulated barrels are configured to receive the primary fixation element and the at least one supplemental fixation element.

3. The device of claim 1, wherein the bone is a calcar of a femur and the device is further configured for affixation in a pertrochanteric region.

4. (canceled)

5. The device of claim 1, wherein the primary fixation element is configured to terminate within a distance to an end of the bone.

6. (canceled)

7. The device of claim 1, wherein the primary fixation element is configured to prevent rotation.

8. (canceled)

9. The device of claim 2, wherein the cannulated barrel configured to receive the primary fixation device, is further configured with a geometry to prevent rotation.

10. The device of claim 2, wherein the cannulated barrel configured to receive the primary fixation device is further configured to be angulated and positioned proximal to a calcar.

11. The device of claim 2, wherein the cannulated barrel configured to receive the primary fixation device is further configured to prevent caudal displacement of a femoral head and femoral neck after insertion of the primary fixation device.

12. The device of claim 1, wherein at least one of: the supplemental fixation element, and the positioning fixation element, comprises a locking or nonlocking bone screw.

13. (canceled)

14. (canceled)

15. The device of claim 2, wherein the cannulated barrel configured to receive the supplemental fixation device is further configured to be angulated so that the supplemental fixation device is positioned for fixation across the fracture.

16. The device of claim 1 wherein the plate is malleable and the tapered end of the plate is configured to be contoured to match the bone.

17. The device of claim 2, wherein the plate is malleable and configured for angulation of the plurality of barrels.

18. (canceled)

19. The device of claim 1 further configured so that the fracture cannot compress more than a set distance once the device is affixed.

20. (canceled)

21. The device of claim 19 further comprising a mechanism configured to prevent femoral neck shortening.

22. (canceled)

23. The device of claim 1, wherein the primary fixation device is configured to couple to a jig for alignment and insertion.

24. The device of claim 1, further comprising a buttress clip with a plurality of holes configured to receive the positioning fixation element or an additional fixation element and configured to resist rotational angulation and translation.

25. The device of claim 24, wherein the buttress clip is further configured for insertion under musculature of a femur, over an anterior cortex of the femur and applied to a caudal aspect of a femoral neck.

26. The device of claim 24, wherein the buttress clip is further configured for insertion without exposure of the fracture site.

27. The device of claim 24, wherein the buttress clip is further configured so that at least one hole of the buttress clip and at least one hole of the plate configured to receive the positioning fixation element align such that the positioning fixation element affixes both the buttress clip and the plate.

28. The device of claim 24, wherein the buttress clip is further configured so that no more than two positioning and additional fixation element axes are within a plane.

29. A jig for affixation of a medical implant comprising,
at least one armature, at least one guide element, and at least one alignment hole;
wherein the at least one armature is configured to couple to the medical implant and the medical implant comprises a plate that tapers at an end and the medical implant is configured for fixation of a bone fracture, to conform to a bone, to receive a primary fixation element, and to receive at least one supplemental fixation element;
wherein the tapered end of the plate is configured with at least one hole to receive at least one positioning fixation element;
wherein the at least one guide element is configured to couple to at least one bone fragment;
wherein the jig is configured to rotate and translate the bone fragment relative to the bone;
wherein rotation and translation of the jig aligns at least one end of the jig with a point on the bone; and
wherein the at least one alignment hole is configured to align a surgical tool with the point on the bone.

30. The jig of claim 29, wherein the medical implant further comprises a plurality of cannulated barrels that extend distally from the plate, and the cannulated barrels are configured to receive the primary fixation element and at least one supplemental fixation element; and
the jig is further configured to align the surgical tool to prepare holes in the bone to accommodate the cannulated barrel.

31. The jig of claim 29, wherein the medical implant further comprises a buttress clip with a plurality of holes configured to receive a positioning fixation element or an additional fixation element and the buttress clip is configured to resist rotational angulation and translation;
wherein the jig is further configured to align the surgical tool to prepare holes in the bone for the fixation of the positioning fixation element and the additional fixation element; and
wherein the jig is further configured to align the positioning fixation element and the additional fixation element with a hole axis.

32. A method of fracture fixation comprising,
aligning a plate with a bone for fracture fixation;
preparing holes in the bone for a plurality of fixation elements;
conforming the plate to the bone for fixation;
aligning a primary fixation element with a primary fixation axis;
inserting a primary fixation element through the plate and into the bone;
aligning a supplemental fixation element with a supplemental fixation axis;
inserting a supplemental fixation element through the plate and into the bone;
applying compression force across a fracture with at least one supplemental fixation element;
aligning and inserting at least one positioning fixation element; and
securing the plate to the bone with the at least one positioning fixation element.

33. The method of claim 32, further comprising positioning a jig to align a surgical tool with at least one point on the bone and preparing holes in the bone with the surgical tool in the bone at the at least one point for the fixation elements.

34. The method of claim 32, further comprising affixing a buttress clip configured to resist rotational angulation and translation.

35. The method of claim 32, wherein the primary fixation element is configured to function as an intermediary buttress to resist rotation, angulation, and shear and for insertion without angulation or rotation.

36. The method of claim 33, further comprising,
coupling a guide element to a first portion of a bone along a first axis on a first side of the fracture;
positioning the jig to align a first jig hole with the first axis;
translating the jig proximal to the bone so the guide element passes through the first jig hole;
positioning the jig to align a second jig hole with a second portion of the bone along a second axis on the second side of the fracture;
preparing at least one hole in the second portion of the bone through the second jig hole;
translating the at least one guide element through the second jig hole and coupling the second at least one guide element to the second portion of the bone;
translating the jig to align the first side of the fracture and the second side of the fracture;
rotating the jig to align the first side of the fracture and the second side of the fracture;
aligning a plurality of fixation device holes with the bone;
preparing a plurality of fixation device holes into the first portion of the bone and the second portion of the bone through the plurality of fixation device holes in the jig;
securing the fixation device to the bone.

37. The method of claim 36, further comprising,
inserting a buttress clip through a passageway in the jig;
positioning the buttress clip on the bone;
coupling a buttress clip fixation element to the bone;
securing the buttress clip to the bone.

38. (canceled)

39. The method of claim 37, further comprising,
aligning a buttress clip fixation guide element with the buttress clip;
passing the buttress clip fixation element through the buttress clip fixation element guide.
